(19) United States
(12) Patent Application Publication
(10) Pub. No.: US 2025/0268862 A1
(43) Pub. Date: Aug. 28, 2025

(54) POTASSIUM-COMPETITIVE ACID BLOCKERS FOR THE TREATMENT OF PATHOLOGICAL HYPERSECRETORY CONDITIONS

(71) Applicant: Daewoong Pharmaceutical Co., Ltd., Seoul (KR)

(72) Inventors: Cyril De Colle, Woburn, MA (US); Paul Rogers, Woburn, MA (US)

(21) Appl. No.: 18/859,206

(22) PCT Filed: Apr. 24, 2023
(86) PCT No.: PCT/US2023/019630

§ 371 Date: Oct. 23, 2024

Related U.S. Application Data
(60) Provisional application No. 63/334,475, filed on Apr. 25, 2022.

(51) Int. Cl. A61K 31/4015

(52) U.S. Cl. CPC A61K 31/4015

(57) ABSTRACT

The present application relates to compounds of formula (I) or (Ia) (e.g., fexuprazan), for treating pathological hypersecretory conditions, such as a condition comprising gastric acid hypersecretion (e.g., Zollinger-Ellison syndrome, idiopathic gastric acid hypersecretion, and/or hypergastrinemia).

### RELATED APPLICATIONS

This application claims the benefit of priority to U.S. Provisional Patent Application No. 63/334,475, filed Apr. 25, 2022, which application is hereby incorporated by reference in its entirety.

### BACKGROUND

Pathological hypersecretory conditions are characterized by excessive and abnormal secretion of a substance made by the body, such as gastric acid. Zollinger-Ellison syndrome is a rare condition in which tumors formed in the duodenum or the pancreas secrete excessive amounts of the gastrin hormone, ultimately leading to overproduction of acid in the stomach. (https://www.niddk.nih.gov/health-information/digestive-diseases/zollinger-ellison-syndrome) Excessive stomach acid caused by Zollinger-Ellison syndrome can lead to further conditions (e.g., peptic ulcer disease, GERD, upper digestive tract bleeding, esophagitis, etc.), underscoring the need to treat the disease early and effectively.

The first line of treatment for Zollinger-Ellison syndrome has been proton pump inhibitors (PPIs). (Ito, T., et al. “Pharmacotherapy of Zollinger-Ellison Syndrome” Expert Opin. Pharmacother. (2013) 14(3):307-321.) The effects and risks of long term use of PPIs, however, is still unknown. Potential concerns with long term use of PPIs include possible malabsorption of vitamin B₁₂, iron, and calcium; hypergastrinemia; pneumonia; dementia; and various drug interactions. (Ambizas, E. M., et al. “Proton Pump Inhibitors: Considerations With Long-Term Use” US Pharm. 2017; 42(7)4-7.) Indeed, the American Gastroenterological Association has recently updated its clinical practice, recommending de-prescribing PPIs. (Targownik L. E., et al. “AGA Clinical Practice Update on De-Prescribing of Proton Pump Inhibitors: Expert Review” Gastroenterology. 2022 Feb. 16:S0016-5085(21)04083-X. doi: 10.1053/j.gastro.2021.12.247. Online ahead of print.) Accordingly, there remains an unmet need for safe and effective treatments for Zollinger-Ellison syndrome.

### Summary of Application

The present application provides a method of treating short bowel syndrome or a pathological hypersecretory condition in a subject, comprising administering to a subject in need thereof an effective amount of a compound of formula (I),

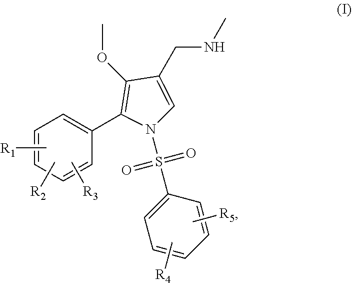

or a pharmaceutically acceptable salt thereof, wherein: R₁, R₂ and R₃ are each independently selected from hydrogen and halogen; R₄ and R₅ are each independently selected from hydrogen, halogen, C₁₋₄alkyl, C₁₋₄alkoxy, C₁₋₄haloalkyl, and C₁₋₄haloalkoxy. In certain such embodiments, the compound of formula (I), or a pharmaceutically acceptable salt thereof, comprises a compound of formula (Ia),

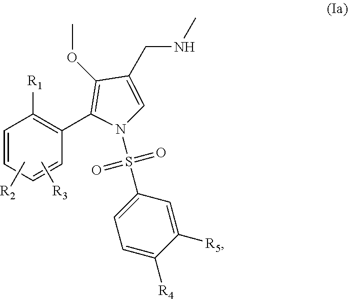

or a pharmaceutically acceptable salt thereof.

In certain embodiments, R₁, R₂ and R₃ are not simultaneously hydrogen. In certain embodiments, R₁, R₂ and R₃ are each independently selected from hydrogen, fluoro, and chloro. In certain embodiments, R₁ is halogen, and R₂ and R₃ are each independently selected from hydrogen and halogen. In certain embodiments, R₁ is fluoro, and R₂ and R₃ are each independently selected from hydrogen, fluoro, and chloro. In certain embodiments, R₁ is chloro, and R₂ and R₃ are each independently hydrogen.

In certain embodiments, R₄ and R₅ are not simultaneously hydrogen. In certain embodiments, R₄ and R₅ are each independently selected from hydrogen, chloro, fluoro, methyl, trifluoromethyl, methoxy, and difluoromethoxy. In certain embodiments, R₄ is hydrogen and R₅ is selected from chloro, fluoro, methyl, trifluoromethyl, methoxy, and difluoromethoxy. In certain embodiments, R₄ and R₅ are each independently selected from chloro and fluoro.

In certain embodiments, wherein R₁ is fluoro, R₂ and R₃ are each independently selected from hydrogen and fluoro, R₄ is hydrogen, and R₅ is selected from chloro and trifluoromethyl.

The present application provides a method of treating short bowel syndrome or a pathological hypersecretory condition in a subject, comprising administering to a subject in need thereof an effective amount of fexuprazan,

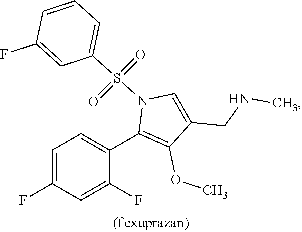 [SMILES: CNCc1cn(S(=O)(=O)c2cccc(F)c2)c(-c2ccc(F)cc2F)c1OC]

or a pharmaceutically acceptable salt thereof. In certain embodiments, the fexuprazan, or a pharmaceutically acceptable salt thereof, is fexuprazan hydrochloride. In certain embodiments, the pathological hypersecretory condition comprises gastric acid hypersecretion. In certain embodiments, the pathological hypersecretory condition is Zollinger-Ellison syndrome, idiopathic gastric acid hypersecretion, and/or hypergastrinemia. In certain embodiments, the pathological hypersecretory condition is Zollinger-Ellison syndrome. In certain embodiments, the pathological hypersecretory condition is idiopathic gastric acid hypersecretion. In certain embodiments, the pathological hypersecretory condition is hypergastrinemia.

The present application provides use of a compound of compound of formula (I),

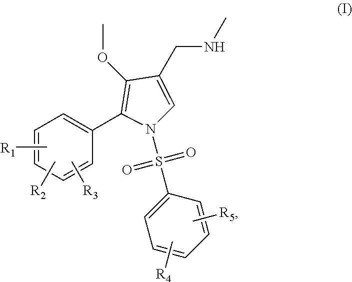

or a pharmaceutically acceptable salt thereof, wherein: R₁, R₂ and R₃ are each independently selected from hydrogen and halogen; and R₄ and R₅ are each independently selected from hydrogen, halogen, C₁₋₄alkyl, C₁₋₄alkoxy, C₁₋₄haloalkyl, and C₁₋₄haloalkoxy; in the preparation of a medicament for the treatment of short bowel syndrome or a pathological hypersecretory condition, such as a condition comprising gastric acid hypersecretion (e.g., Zollinger-Ellison syndrome, idiopathic gastric acid hypersecretion, and/or hypergastrinemia). In certain embodiments, the pathological hypersecretory condition comprises gastric acid hypersecretion. In certain embodiments, the pathological hypersecretory condition is Zollinger-Ellison syndrome. In certain embodiments, the pathological hypersecretory condition is idiopathic gastric acid hypersecretion. In certain embodiments, the pathological hypersecretory condition is hypergastrinemia.

### DETAILED DESCRIPTION OF THE APPLICATION

The present application provides a method of treating short bowel syndrome or a pathological hypersecretory condition, such as a condition comprising gastric acid hypersecretion (e.g., Zollinger-Ellison syndrome, idiopathic gastric acid hypersecretion, and/or hypergastrinemia) in a subject, the method comprising administering to the subject a pharmaceutical composition comprising a potassium-competitive acid blocker (P-CAB). In certain such embodiments, the P-CAB comprises a compound of formula (I),

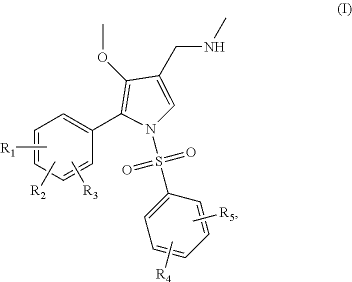

or a pharmaceutically acceptable salt thereof, wherein:
R₁, R₂ and R₃ are each independently hydrogen, or halogen,
R₄ and R₅ are each independently hydrogen, halogen, C₁₋₄alkyl, C₁₋₄alkoxy, C₁₋₄haloalkyl, or C₁₋₄haloalkoxy.

In certain embodiments, R₁, R₂ and R₃ are not simultaneously hydrogen.

In certain embodiments, R₄ and R₅ are not simultaneously hydrogen.

In certain embodiments, R₁, R₂ and R₃ are each independently hydrogen, fluoro, or chloro.

In certain embodiments, R₄ and R₅ are each independently hydrogen, chloro, fluoro, methyl, trifluoromethyl, methoxy, or difluoromethoxy.

The present application provides a method of treating short bowel syndrome or a pathological hypersecretory condition, such as a condition comprising gastric acid hypersecretion (e.g., Zollinger-Ellison syndrome, idiopathic gastric acid hypersecretion, and/or hypergastrinemia) in a subject, the method comprising administering to the subject a pharmaceutical composition comprising a compound of formula (Ia),

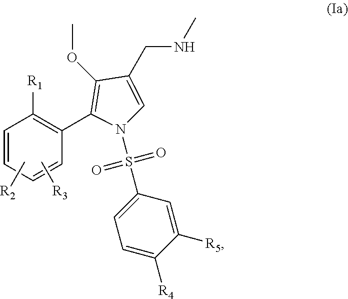

or a pharmaceutically acceptable salt thereof, wherein:
R₁, R₂ and R₃ are each independently hydrogen, or halogen,
R₄ and R₅ are each independently hydrogen, halogen, C₁₋₄alkyl, C₁₋₄alkoxy, C₁₋₄haloalkyl, or C₁₋₄haloalkoxy.

In certain embodiments, R₁, R₂ and R₃ are not simultaneously hydrogen.

In certain embodiments, R₄ and R₅ are not simultaneously hydrogen.

In certain embodiments, R₁, R₂ and R₃ are each independently hydrogen, fluoro, or chloro. In certain embodiments, R₁ is halogen, and R₂ and R₃ are each independently hydrogen, or halogen. In certain embodiments, R₁ is fluoro, and R₂ and R₃ are each independently hydrogen, fluoro, or chloro; or R₁ is chloro and R₂ and R₃ may be hydrogen.

In certain embodiments, R₄ and R₅ are each independently hydrogen, chloro, fluoro, methyl, trifluoromethyl, methoxy, or difluoromethoxy. In certain embodiments, R₄ is hydrogen, R₅ is chloro, fluoro, methyl, trifluoromethyl, methoxy, or difluoromethoxy; or R₄ and R₅ may be each independently chloro or fluoro.

In certain embodiments, R₁ is fluoro, and R₂ and R₃ are each independently hydrogen, or fluoro, R₄ is hydrogen, and R₅ may be chloro, or trifluoromethyl.

The present application provides a method of treating short bowel syndrome or a pathological hypersecretory condition, such as a condition comprising gastric acid hypersecretion (e.g., Zollinger-Ellison syndrome, idiopathic gastric acid hypersecretion, and/or hypergastrinemia) in a subject, the method comprising administering to the subject a pharmaceutical composition comprising fexuprazan,

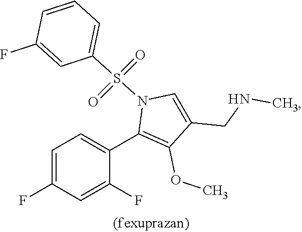 [SMILES: CNCc1cn(S(=O)(=O)c2cccc(F)c2)c(-c2ccc(F)cc2F)c1OC]

or a pharmaceutically acceptable salt thereof. In certain embodiments, the fexuprazan, or a pharmaceutically acceptable salt thereof, is fexuprazan hydrochloride. In certain embodiments, the fexuprazan, or a pharmaceutically acceptable salt thereof, is fexuprazan succinate. In certain embodiments, the fexuprazan, or a pharmaceutically acceptable salt thereof, is fexuprazan tartrate. In certain embodiments, the fexuprazan, or a pharmaceutically acceptable salt thereof, is fexuprazan fumarate.

In certain embodiments, administration of the compound of formula (I) or (Ia) (e.g., fexuprazan), or a pharmaceutically acceptable salt of the compound according to formula (I) or (Ia) (e.g., fexuprazan) for the treatment of short bowel syndrome or a pathological hypersecretory condition, such as a condition comprising gastric acid hypersecretion (e.g., Zollinger-Ellison syndrome, idiopathic gastric acid hypersecretion, and/or hypergastrinemia) results in a reduction of upper gastrointestinal bleeding. The compounds of formula (I) or (Ia) (e.g., fexuprazan), or a pharmaceutically acceptable salt thereof, have proven effective at inhibiting gastric damage. See, e.g., U.S. Pat. No. 10,100,010.

In certain embodiments of the methods disclosed herein comprising administering to the subject a pharmaceutical composition comprising fexuprazan, or a pharmaceutically acceptable salt thereof, the fexuprazan, or a pharmaceutically acceptable salt thereof, comprises a crystalline form of fexuprazan, or a pharmaceutically acceptable salt thereof. Certain such crystalline forms of fexuprazan, or a pharmaceutically acceptable salt thereof, are disclosed in, e.g., U.S. Pat. No. 10,336,695.

In certain embodiments, the crystalline form of fexuprazan, or a pharmaceutically acceptable salt thereof, as used in the methods disclosed herein, is prepared as disclosed in, e.g., U.S. Pat. No. 10,336,695. For example, the crystalline form of fexuprazan, or a pharmaceutically acceptable salt thereof, can be prepared by various crystallization methods such as an evaporative crystallization method, a drowning-out crystallization method, a reactive crystallization method, a solvent-mediated polymorphic transition method, and a solid-state polymorphic transition method, which are selected according to the thermodynamic and dynamic characteristics of the salt.

In certain embodiments, the crystalline form of fexuprazan, or a pharmaceutically acceptable salt thereof, as used in the methods disclosed herein, can be identified through an X-ray powder diffraction analysis and/or a differential scanning calorimetry analysis. As an example, the crystalline form of fexuprazan, or a pharmaceutically acceptable salt thereof, can be classified through a diffraction angle (2θ) exhibiting a characteristic peak in an X-ray powder diffraction pattern and/or an intensity of a peak according to the respective diffraction angles (2θ). In certain such embodiments, the diffraction angle (2θ) can be varied by ±0.2° or preferably ±0.1° due to various factors such as a manufacturing technique of the measurement sample, a fixing procedure of the measurement sample, and a measuring instrument.

In certain embodiments, the crystalline form of fexuprazan, or a pharmaceutically acceptable salt thereof, as used in the methods disclosed herein, can be identified through the endothermic initiation temperature and the endothermic temperature indicating the maximum endothermic peak in the differential scanning calorimetry analysis. In certain such embodiments, the temperature may be varied by ±3° C., preferably ±2° C., or more preferably ±1° C., depending on various factors such as a manufacturing technique of the measurement sample, a measuring instrument, and a rate of temperature change.

In certain embodiments, the crystalline form of fexuprazan, or a pharmaceutically acceptable salt thereof, as used in the methods disclosed herein, comprises crystalline form I of fexuprazan hydrochloride, wherein crystalline form I of fexuprazan hydrochloride is characterized by an X-ray powder diffraction pattern comprising two or more, three or more, four or more, or five or more of the following 2θ values: 5.8°, 9.7°, 10.0°, 12.8°, 13.2°, 17.4° and 18.5°, ±0.2°. In certain embodiments, the crystalline form of fexuprazan, or a pharmaceutically acceptable salt thereof, as used in the methods disclosed herein, comprises crystalline form I of fexuprazan hydrochloride, wherein crystalline form I of fexuprazan hydrochloride is characterized by an X-ray powder diffraction pattern comprising two or more, three or more, four or more, or five or more of the following 2θ values: 5.8°, 9.7°, 10.0°, 12.8°, 13.2°, 17.4°, 18.5°, 19.5°, 19.8°, 20.1°, 21.8°, 25.9°, 26.5° and 28.2°±0.2°. In certain embodiments, the crystalline form of fexuprazan, or a pharmaceutically acceptable salt thereof, as used in the methods disclosed herein, comprises crystalline form I of fexuprazan hydrochloride, wherein crystalline form I of fexuprazan hydrochloride is characterized by an X-ray powder diffraction pattern comprising two or more, three or more, four or more, or five or more of the following 2θ values: 5.8°, 9.7°, 10.0°, 12.8°, 13.2°, 17.4°, 18.5°, 19.5°, 19.8°, 20.1°, 25.9° and 28.2°, ±0.20.

In certain embodiments, the crystalline form of fexuprazan, or a pharmaceutically acceptable salt thereof, as used in the methods disclosed herein, comprises crystalline form I of fexuprazan hydrochloride, wherein crystalline form I of fexuprazan hydrochloride is characterized by an endothermic initiation temperature of 215.02±3° C. and exhibit a maximum endothermic peak at an endothermic temperature of 217.11±3° C. in a differential scanning calorimetry analysis.

In certain embodiments, the crystalline form of fexuprazan, or a pharmaceutically acceptable salt thereof, as used in the methods disclosed herein, comprises crystalline form II of fexuprazan hydrochloride, wherein crystalline form II of fexuprazan hydrochloride is characterized by an X-ray powder diffraction pattern comprising two or more, three or more, or four of the following 2θ values: 9.2°, 10.0°, 12.9° and 20.2°, ±0.2°. In certain embodiments, the crystalline form of fexuprazan, or a pharmaceutically acceptable salt thereof, as used in the methods disclosed herein, comprises crystalline form II of fexuprazan hydrochloride, wherein crystalline form II of fexuprazan hydrochloride is characterized by an X-ray powder diffraction pattern comprising two or more, three or more, four or more, or five or more of the following 2θ values: 9.2°, 9.8°, 10.0°, 12.9°, 13.2°, 13.4°, 13.8°, 15.0°, 18.4°, 19.6° and 20.2°±0.2°.

In certain embodiments, the crystalline form of fexuprazan, or a pharmaceutically acceptable salt thereof, as used in the methods disclosed herein, comprises crystalline form II of fexuprazan hydrochloride, wherein crystalline form II of fexuprazan hydrochloride is characterized by an endothermic initiation temperature of 213.14±3° C. and exhibit a maximum endothermic peak at an endothermic temperature of 215.7±3° C. in a differential scanning calorimetry analysis.

In certain embodiments, the crystalline form of fexuprazan, or a pharmaceutically acceptable salt thereof, as used in the methods disclosed herein, comprises a crystalline form of fexuprazan succinate, wherein the crystalline form of fexuprazan succinate is characterized by an X-ray powder diffraction pattern comprising two or more, three or more, four or more, or five or more of the following 2θ values: 8.0°, 11.2°, 12.0°, 14.9°, 22.1° and 24.1°, ±0.2°. In certain embodiments, the crystalline form of fexuprazan, or a pharmaceutically acceptable salt thereof, as used in the methods disclosed herein, comprises a crystalline form of fexuprazan succinate, wherein the crystalline form of fexuprazan succinate is characterized by an X-ray powder diffraction pattern comprising two or more, three or more, four or more, or five or more of the following 2θ values: 8.0°, 11.2°, 12.0°, 14.9°, 20.0°, 22.1° and 24.1°±0.2°.

In certain embodiments, the crystalline form of fexuprazan, or a pharmaceutically acceptable salt thereof, as used in the methods disclosed herein, comprises a crystalline form of fexuprazan succinate, wherein the crystalline form of fexuprazan succinate is characterized by an endothermic initiation temperature of 132.3±3° C. and exhibit a maximum endothermic peak at an endothermic temperature of 133.9±3° C. in a differential scanning calorimetry analysis.

In certain embodiments, the crystalline form of fexuprazan, or a pharmaceutically acceptable salt thereof, as used in the methods disclosed herein, comprises a crystalline form of fexuprazan tartrate, wherein the crystalline form of fexuprazan tartrate is characterized by an X-ray powder diffraction pattern comprising two or more or three of the following 2θ values: 11.7°, 21.5° and 23.5°, ±0.2°. In certain embodiments, the crystalline form of fexuprazan, or a pharmaceutically acceptable salt thereof, as used in the methods disclosed herein, comprises a crystalline form of fexuprazan tartrate, wherein the crystalline form of fexuprazan tartrate is characterized by an X-ray powder diffraction pattern comprising two or more, three or more, four or more, or five or more of the following 2θ values: 11.7°, 13.0°, 13.5°, 14.5°, 18.3°, 19.5°, 20.3°, 21.5° and 23.5°±0.20.

In certain embodiments, the crystalline form of fexuprazan, or a pharmaceutically acceptable salt thereof, as used in the methods disclosed herein, comprises a crystalline form of fexuprazan tartrate, wherein the crystalline form of fexuprazan tartrate is characterized by an endothermic initiation temperature of 146.34±3° C. and exhibit a maximum endothermic peak at an endothermic temperature of 148.27° C. in a differential scanning calorimetry analysis.

In certain embodiments, the crystalline form of fexuprazan, or a pharmaceutically acceptable salt thereof, as used in the methods disclosed herein, comprises crystalline form I of fexuprazan fumarate, wherein crystalline form I of fexuprazan fumarate is characterized by an X-ray powder diffraction pattern comprising two or more or three of the following 2θ values: 7.9°, 11.9° and 24.0°, ±0.2°. In certain embodiments, the crystalline form of fexuprazan, or a pharmaceutically acceptable salt thereof, as used in the methods disclosed herein, comprises crystalline form I of fexuprazan fumarate, wherein crystalline form I of fexuprazan fumarate is characterized by an X-ray powder diffraction pattern comprising two or more, three or more, or four of the following 2θ values: 7.9°, 11.9°, 20.0° and 24.0°±0.2°.

In certain embodiments, the crystalline form of fexuprazan, or a pharmaceutically acceptable salt thereof, as used in the methods disclosed herein, comprises crystalline form I of fexuprazan fumarate, wherein crystalline form I of fexuprazan fumarate is characterized by an endothermic initiation temperature of 164.97±3° C. and exhibit a maximum endothermic peak at an endothermic temperature of 167.46±3° C. in a differential scanning calorimetry analysis.

In certain embodiments, the crystalline form of fexuprazan, or a pharmaceutically acceptable salt thereof, as used in the methods disclosed herein, comprises crystalline form II of fexuprazan fumarate, wherein crystalline form II of fexuprazan fumarate is characterized by an X-ray powder diffraction pattern comprising two or more, three or more, or four of the following 2θ values: 8.4°, 10.5°, 18.3° and 19.02°±0.20.

In certain embodiments, the crystalline form of fexuprazan, or a pharmaceutically acceptable salt thereof, as used in the methods disclosed herein, comprises crystalline form II of fexuprazan fumarate, wherein crystalline form II of fexuprazan fumarate is characterized by an endothermic initiation temperature of 179.47±3° C. and exhibit a maximum endothermic peak at an endothermic temperature of 189.05° C. in a differential scanning calorimetry analysis.

In certain embodiments wherein alkyl, alkenyl, alkynyl, alkoxy, aryl, heteroaryl, cycloalkyl, heterocycloalkyl, or oxime are substituted, they are substituted, valency permitting, with one or more substituents selected from substituted or unsubstituted alkyl, such as perfluoroalkyl (e.g., trifluoromethyl), alkenyl, alkoxy, alkoxyalkyl, aryl, aralkyl, arylalkoxy, aryloxy, aryloxyalkyl, hydroxyl, halo, alkoxy, such as perfluoroalkoxy (e.g., trifluoromethoxy), alkoxyalkoxy, hydroxyalkyl, hydroxyalkylamino, hydroxyalkoxy, amino, aminoalkyl, alkylamino, aminoalkylalkoxy, aminoalkoxy, acylamino, acylaminoalkyl, such as perfluoro acylaminoalkyl (e.g., trifluoromethylacylaminoalkyl), acyloxy, cycloalkyl, cycloalkylalkyl, cycloalkylalkoxy, heterocyclyl, heterocyclylalkyl, heterocyclyloxy, heterocyclylalkoxy, heteroaryl, heteroarylalkyl, heteroarylalkoxy, heteroaryloxy, heteroaryloxyalkyl, heterocyclylaminoalkyl, heterocyclylaminoalkoxy, amido, amidoalkyl, amidine, imine, oxo, carbonyl (such as carboxyl, alkoxycarbonyl, formyl, or acyl, including perfluoroacyl (e.g., C(O)CF₃)), carbonylalkyl (such as carboxyalkyl, alkoxycarbonylalkyl, formylalkyl, or acylalkyl, including perfluoroacylalkyl (e.g., -alkylC(O)CF₃)), carbamate, carbamatealkyl, urea, ureaalkyl, sulfate, sulfonate, sulfamoyl, sulfone, sulfonamide, sulfonamidealkyl, cyano, nitro, azido, sulfhydryl, alkylthio, thiocarbonyl (such as thioester, thioacetate, or thioformate), phosphoryl, phosphate, phosphonate or phosphinate.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in chemically stable compounds.

As used in this application, the term “optionally substituted” means that substitution is optional and therefore it is possible for the designated atom or moiety to be unsubstituted.

Compounds of the present application containing one or multiple asymmetrically substituted atoms may be isolated in optically active or racemic forms. It is well known in the art how to prepare optically active forms, such as by resolution of racemic forms, by synthesis from optically active starting materials, or by synthesis using optically active reagents.

In certain embodiments, compounds of the application may be racemic. For example, in embodiments of the application wherein a compound (e.g., a compound of formula (I) or (Ia) (e.g., fexuprazan), or a pharmaceutically acceptable salts thereof) is disclosed herein as a particular enantiomer, the application further contemplates the compound in its racemic form. In certain embodiments, compounds of the application may be enriched in one enantiomer. For example, a compound of the application may have greater than 30% ee, 40% ee, 50% ee, 60% ee, 70% ee, 80% ee, 90% ee, or even 95% or greater ee.

In certain embodiments, the therapeutic preparation may be enriched to provide predominantly one enantiomer of a compound (e.g., of formula (I) or (Ia) (e.g., fexuprazan), or a pharmaceutically acceptable salt thereof). An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, compounds of the application may have more than one stereocenter. In certain such embodiments, compounds of the application may be enriched in one or more diastereomer. For example, a compound of the application may have greater than 30% de, 40% de, 50% de, 60% de, 70% de, 80% de, 90% de, or even 95% or greater de.

In certain embodiments, the therapeutic preparation may be enriched to provide predominantly one diastereomer of a compound (e.g., of formula (I) or (Ia) (e.g., fexuprazan), or a pharmaceutically acceptable salt thereof). A diastereomerically enriched mixture may comprise, for example, at least 60 mol percent of one diastereomer, or more preferably at least 75, 90, 95, or even 99 mol percent.

A variety of compounds in the present application may exist in particular geometric or stereoisomeric forms. The present application takes into account all such compounds, including tautomers, cis- and trans-isomers, R- and S-enantiomers, diastereomers, (D)-isomers, (L)-isomers, the racemic mixtures thereof, and other mixtures thereof, as being covered within the scope of this application. All tautomeric forms are encompassed in the present application. Additional asymmetric carbon atoms may be present in a substituent such as an alkyl group. All such isomers, as well as mixtures thereof, are intended to be included in this application, unless the stereochemistry or isomeric form is specifically indicated.

The present application further includes all pharmaceutically acceptable isotopically labelled compounds (e.g., of formula (I) or (Ia) (e.g., fexuprazan), or pharmaceutically acceptable salts thereof). An “isotopically” or “radio-labelled” compound is a compound where one or more atoms are replaced or substituted by an atom having an atomic mass or mass number different from the atomic mass or mass number typically found in nature (i.e., naturally occurring). For example, in certain embodiments, in compounds (e.g., of formula (I) or (Ia) (e.g., fexuprazan), or pharmaceutically acceptable salts thereof), hydrogen atoms are replaced or substituted by one or more deuterium or tritium (e.g., hydrogen atoms on a C₁₋₆ alkyl or a C₁₋₆ alkoxy are replaced with deuterium, such as d₃-methoxy or 1,1,2,2-d₄-3-methylbutyl).

Certain isotopically labelled compounds (e.g., of formula (I) or (Ia) (e.g., fexuprazan), or pharmaceutically acceptable salts thereof), in the application, for example, those incorporating a radioactive isotope, are useful in drug and/or substrate tissue distribution studies. The radioactive isotopes tritium, i.e. ³H, and carbon 14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy.

Isotopically labelled compounds (e.g., of formula (I) or (Ia) (e.g., fexuprazan), or pharmaceutically acceptable salts thereof) can generally be prepared by conventional techniques known to those skilled in the art or by processes analogous to those described in the accompanying examples using an appropriate isotopically labelled reagent in place of the non-labelled reagent previously employed. Suitable isotopes that may be incorporated in compounds of the present application include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I.

In certain embodiments, the present application provides a pharmaceutical preparation suitable for use in a human patient, comprising any of the compounds shown above (e.g., a compound of the application, such as a compound of formula (I) or (Ia) (e.g., fexuprazan), or pharmaceutically acceptable salts thereof) and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### Uses of the Compounds

Compounds of the present application may be administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient. The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. This, the skilled artisan can readily determine the amount of compound and optional additives, vehicles, and/or carrier in compositions and to be administered in methods of the application. In certain embodiments, the application relates to a compound according to formula (I) or (Ia) (e.g., fexuprazan), or a pharmaceutically acceptable salt of the compound according to formula (I) or (Ia) (e.g., fexuprazan), for use as a medicament, e.g., for treatment of any of the disorders disclosed herein.

In certain embodiments, the application relates to the use of a compound according to formula (I) or (Ia) (e.g., fexuprazan), or a pharmaceutically acceptable salt of the compound according to formula (I) or (Ia) (e.g., fexuprazan), in the manufacture of a medicament for treating short bowel syndrome or a pathological hypersecretory condition, such as a condition comprising gastric acid hypersecretion (e.g., Zollinger-Ellison syndrome, idiopathic gastric acid hypersecretion, and/or hypergastrinemia).

In the treatment of any of the disorders disclosed herein, different compounds of the application may be (e.g., conjointly) administered with one or more other compounds of the application. Moreover, compounds of formula (I) or (Ia) (e.g., fexuprazan), or a pharmaceutically acceptable salt of the compound of formula (I) or (Ia) (e.g., fexuprazan), or certain combinations thereof, may be conjointly administered with other conventional therapeutic agents in treating one or more disease conditions referred to herein.

In certain embodiments, compounds of the application may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously, sequentially, or by separate dosing of the individual components of the treatment. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the application with one or more additional therapeutic agent(s) provides improved efficacy relative to each individual administration of the compound of the application (e.g., compound of formula (I) or (Ia) (e.g., fexuprazan), or a pharmaceutically acceptable salt of the compound of formula (I) or (Ia) (e.g., fexuprazan)) or the one or more additional therapeutic agent(s). In certain such embodiments, the conjoint administration provides an additive effect, wherein an additive effect refers to the sum of each of the effects of individual administration of the compound of the application and the one or more additional therapeutic agent(s).

Additional agents may be: small molecules, nutraceuticals, vitamins, e.g., vitamin D, drugs, pro-drugs, biologics, peptides, peptide mimetics, antibodies, antibody fragments, cell or tissue transplants, vaccines, polynucleotides, DNA molecules, RNA molecules, (i.e. -siRNA, miRNA), antibodies conjugated to drugs, toxins, fusion proteins. Agents may be delivered by vectors, including but not limited to: plasmid vectors, viral vectors, non-viral vectors, liposomal formulations, nanoparticle formulations, toxins, therapeutic radioisotopes, etc.

The additional agent can be an agent for use in the treatment of an enteric nervous system disorder. In some embodiments, the additional agent is an anti-emetic agent (e.g., used for the treatment of nausea and/or vomiting). The additional anti-emetic agent can be, by way of non-limiting example only, a 5-HT3 receptor antagonist, a dopamine receptor antagonist, an NK1 receptor antagonist, an antihistamine, a cannabinoid, a benzodiazepine, an anticholinergic agent, a steroid, or other anti-emetics. Exemplary 5-HT3 receptor antagonists include, but are not limited to, Odansetron, Tropisetron, Granisetron, Palonosetron, Dolasetron. Exemplary dopamine receptor antagonists include, e.g., metopimazine and pharmaceutically acceptable salts thereof (e.g., metopimazine mesylate), Metoclopramide (Reglan), Domperidone (Motilium), Olanzapine (Zyprexa) Droperidol, haloperidol, chlorpromazine, promethazine, prochlorperazine, Alizapride, Prochlorperazine, Sulpiride. Exemplary NK1 receptor antagonists include, e.g., Aprepitant, Tradipitant or Casopitant. Exemplary antihistamines include, e.g., Cyclizine, Diphenhydramine (Benadryl), Dimenhydrinate (Gravol, Dramamine), Doxylamine, Meclozine (Bonine, Antivert), Promethazine (Pentazine, Phenergan, Promacot), and Hydroxyzine (Vistaril), Cimetidine, Famotidine, Lafutidine, Nizatidine, Ranitidine, Roxatidine, Tiotidine. Exemplary cannabinoids include, e.g., Cannabis, Sativex, tetrahydrocannabinol, Dronabinol, and synthetic cannabinoids such as Nabilone.

Exemplary benzodiazepines include, e.g., midazolam or lorazepam. Exemplary anticholinergic agents include, e.g., scopolamine. Other exemplary anti-emetics include, e.g., Trimethobenzamide, Ginger, Emetrol, Propofol, Peppermint, erythromycin, Muscimol, botulinum toxin A (e.g., injected into the stomach to relax the pyloric muscle), and Ajwain.

The additional agent can be an agent for treatment of a disease or clinical syndrome associated with gastroparesis. Exemplary other diseases and clinical syndromes are described herein. The additional agent can be an agent for treatment of diabetes. Exemplary agents for the treatment of diabetes include, e.g., insulin. Other agents for the treatment of diabetes are described in, for example, U.S. Pat. Nos. 6,274,549, 8,349,818, 6,184,209, US Patent Application Publication No. US20070129307, and PCT Application Publication No. WO/2004/082667A1, all of which are hereby incorporated by reference.

The additional agent can be for treatment of upper and lower dysmotility disorders associated with Parkinson's disease. The additional agent can be for treatment of Parkinson's disease. Exemplary agents for the treatment of Parkinson's disease include, e.g., dopaminergic agents, MAO-A or B inhibitors such as, e.g., selegiline, COMT inhibitors such as entacapone, amantadine, stem cell transplant, and neuroprotective agents. Exemplary dopaminergic agents include, but are not limited to levodopa, bromocriptine, pergolide, pramipexole, cabergoline, ropinorole, apomorphine or a combination thereof.

The additional agent can be for treatment of hypothyroidism, hyperthyroidism, or hyperparathyroidism. Exemplary agents for the treatment of such diseases include, e.g., beta-adrenergic blockers (“beta blockers”), levothyroxine calcimimetics, estrogen, progesterone, bisphosphonates.

The additional agent can be for treatment of adrenal insufficiency. Exemplary agents for treatment of adrenal insufficiency include, e.g., corticosteroid hormones (for example, aldosterone, fludrocortisones, and cortisol).

The additional agent can be for treatment of gastroesophageal reflux. Exemplary agents for treatment of gastroesophageal reflux include, e.g., antacids such as, for example, proton pump inhibitors such as omeprazole, H2 receptor antagonists such as ranitidine, antacids, mosapride, sucralfate, and baclofen.

The additional agent can be for treatment of scleroderma. For example, the additional agent can be D-penicillamine, colchicine, PUVA, relaxin, cyclosporine, and EPA (omega-3 oil derivative), immunosupressants such as, e.g., methotrexate, cyclophosphamide, azathioprine, and mycophenolate. The additional agent can be for treatment of polymyositis. For example, the additional agent can be a corticosteroid, e.g., prednisone, or can be an immunosuppressant.

The additional agent can be for treatment of muscular dystrophy. For example, the additional agent can be, e.g., a glucocorticoid receptor antagonist. Exemplary glucocorticoid receptor antagonists include, but are not limited to, mifepristone, 113-(4-dimethylaminoethoxyphenyl)-17α-propynyl-17p-hydroxy-4,9 estradien-3-one, 17β-hydroxy-17α-19-(4-methylphenyl)androsta-4,9(11)-dien-3-one, 4α(S)-Benzyl-2(R)-prop-1-ynyl-1,2,3,4,4α,9,10,10α(R)-octahydro-phenanthrene-2,7-diol and 4α(S)-Benzyl-2(R)-chloroethynyl-1,2,3,4,4α,9,10,10α(R)-octahydro-phenanthrene-2,7-diol, (11β,17β)-11-(1,3-benzodioxo-5-yl)-17-hydroxy-17-(1-propynyl)estra-4,9-dien-3-one.

The additional agent can be for treatment of amyloidosis. For example, the additional agent can be an amyloid beta sheet mimic, an antioxidant, molecular chaperone, or other agent. Exemplary agents for the treatment of amyloidosis are described in, e.g., WO/2008/141074. Exemplary molecular chaperones include, e.g., HSP60, HSP70, HSP90, HSP100, BiP, GRP94, GRP170, calnexin and calreticulin, Protein disulfide isomerase (PDI), Peptidyl prolyl cis-trans-isomerase (PPI), trimethylamine N-oxide (TMAO), betaine, glycine betaine, glycero-phosphorylcholine, carbohydrates such as, e.g., glycerol, sorbitol, arabitol, myo-inositol and trehalose, choline, 4-Phenyl butyric acid, and taurine-conjugated ursodeoxycholic acid.

The additional agent can be for treatment of chronic idiopathic pseudoobstruction. For example, the additional agent can be Prucalopride, Pyridostigmine, Metoclopramide, cisapride, linaclotide, octreotide, cannabinoids, and erythromycin.

The additional agent can be for treatment of dermatomyositis. For example, the additional agent can be Prednisolone, Methotrexate, Mycophenolate (CellCept/Myfortic), intravenous immunoglobulins, Azathioprine (Imuran), Cyclophosphamide, Rituximab, and Acthar Gel.

The additional agent can be for treatment of systemic lupus erytematosus. For example, the additional agent can be renal transplant, corticosteroids, immunosupressants, Hydroxychloroquine, Cyclophosphamide, Mycophenolic acid, immunosupressants, analgesics, intravenous immunoglobins, and the like.

The additional agent can be for treatment of anorexia and/or bulimia. For example, the additional agent can be olanzapine, a tricyclic antidepressant, an MAO inhibitor, mianserin, a selective serotonin reuptake inhibitor, e.g., fluoxetine, lithium carbonate, trazodone, and bupropion, phenytoin, carbamazepine, and valproic acid, opiate antagonists such as, e.g., naloxone and naltrexone, and topiramate.

The additional agent can be for treatment of depression. For example, the additional agent can be a selective serotonin reuptake inhibitor, a serotonin and norepinephrine reuptake inhibitor, bupropion, a tricyclic antidepressant, a monoamine oxidase inhibitor, and the like. The additional agent can be for treatment of paraneoplastic syndrome. The additional agent can be for treatment of a high cervical cord lesion. For example, the additional agent can be a corticosteroid or other anti-inflammatory medication. The additional agent can be for treatment of multiple sclerosis. For example, the additional agent can be interferon beta-lb, interferon beta-1a, Glatiramer acetate, Mitoxantrone, natalizumab, fingolimod, teriflunomide, or cladribine.

The additional therapeutic agent can be selected from the group consisting of serotonin agonists, serotonin antagonists, selective serotonin reuptake inhibitors, anticonvulsants, opioid receptor agonists, bradykinin receptor antagonists, NK receptor antagonists, adrenergic receptor agonists, benzodiazepines, gonadotropin-releasing hormone analogues, calcium channel blockers, and somatostatin analogs.

Dosages of the additional agent and of a pharmaceutical composition as described herein for use in the described treatments can vary depending on the type of additional therapeutic agent employed, on the disease or condition being treated and so forth. Sub-therapeutic amounts of one or both of the additional agent and the pharmaceutical composition as described herein can be used. The sub-therapeutic amount of one or both of the additional agent and the pharmaceutical composition as described herein can be a synergistically effective amount. Therapeutically effective amounts of one or both of the additional agent and the pharmaceutical composition as described herein can be used. The pharmaceutical composition as described herein and the additional agent may be administered either simultaneously or sequentially. If administered sequentially, the attending physician or caretaker can decide on the appropriate sequence of administering the pharmaceutical composition as described herein and the additional therapeutic agent.

In some embodiments, a method comprising administering any of the pharmaceutical compositions described herein further comprises combination therapy with an additional therapeutic regimen. The additional therapeutic regimen can comprise implantation of a medical device. The medical device can be implanted in the stomach and/or abdomen, e.g., in the duodenum. The medical device can be an electrical device. The medical device can be a pacemaker. Such a pacemaker can utilize electrical current to induce stomach and/or duodenal contractions, thereby promoting gastrointestinal motility. Such medical devices, and methods of using them, are disclosed in U.S. Pat. No. 8,095,218, hereby incorporated by reference.

### Definitions

The definitions set forth in this application are intended to clarify terms used throughout this application.

The term “herein” means the entire application.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

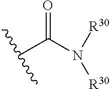

wherein each R³⁰ independently represent a hydrogen or hydrocarbyl group, or two R³⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

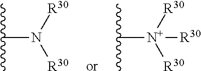

wherein each R³⁰ independently represents a hydrogen or a hydrocarbyl group, or two R³⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

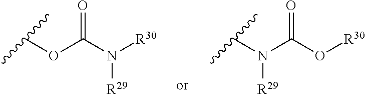

wherein R²⁹ and R³¹ independently represent hydrogen or a hydrocarbyl group, such as an alkyl group, or R²⁹ and R³¹ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂-R³⁰, wherein R³⁰ represents a hydrocarbyl group.

The term “carboxy”, as used herein, refers to a group represented by the formula —CO₂H.

The term “ester”, as used herein, refers to a group —C(O)OR³⁰ wherein R³⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Exemplary heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

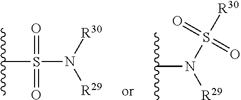

wherein R²⁹ and R³⁰ independently represents hydrogen or hydrocarbyl, such as alkyl, or R²⁹ and R³⁰ taken together with the intervening atom(s) complete a heterocycle having from 4 to 8 atoms in the ring structure.

The term “sulfoxide” is art-recognized and refers to the group —S(O)—R³⁰, wherein R³⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂-R³⁰, wherein R³⁰ represents a hydrocarbyl.

The term “thioalkyl”, as used herein, refers to an alkyl group substituted with a thiol group.

The term “thioester”, as used herein, refers to a group —C(O)SR³⁰ or —SC(O)R³⁰ wherein R³⁰ represents a hydrocarbyl.

The term “thioether”, as used herein, is equivalent to an ether, wherein the oxygen is replaced with a sulfur.

The term “urea” is art-recognized and may be represented by the general

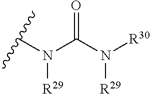

wherein R²⁹ and R³⁰ independently represent hydrogen or a hydrocarbyl, such as alkyl, or either occurrence of R²⁹ taken together with R³⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The present application includes prodrugs of the compounds formula (I) or (Ia) (e.g., fexuprazan), or pharmaceutically acceptable salts thereof. The term “prodrug” is intended to encompass compounds which, under physiologic conditions, are converted into the therapeutically active agents of the present application (e.g., a compound of formula (I) or (Ia) (e.g., fexuprazan), or pharmaceutically acceptable salts thereof). A common method for making a prodrug is to include one or more selected moieties which are hydrolyzed under physiologic conditions to yield the desired molecule. In certain embodiments, the prodrug is converted by an enzymatic activity of the host animal. For example, a prodrug with a nitro group on an aromatic ring could be reduced by reductase to generate the desired amino group of the corresponding active compound in vivo. In another example, functional groups such as a hydroxyl, carbonate, or carboxylic acid in the parent compound are presented as an ester, which could be cleaved by esterases. Additionally, amine groups in the parent compounds are presented in, but not limited to, carbamate, N-alkylated or N-acylated forms (Simplfcio et al, “Prodrugs for Amines,” Molecules, (2008), 13:519-547). In certain embodiments, some or all of the compounds of formula (I) or (Ia) (e.g., fexuprazan), or pharmaceutically acceptable salts thereof, in a formulation represented above can be replaced with the corresponding suitable prodrug.

The present application includes metabolites of the compounds of formula (I) or (Ia) (e.g., fexuprazan), or pharmaceutically acceptable salts thereof. The term “metabolite” is intended to encompass compounds that are produced by metabolism/biochemical modification of the parent compound under physiological conditions, e.g. through certain enzymatic pathway. For example, an oxidative metabolite is formed by oxidation of the parent compound during metabolism, such as the oxidation of a pyridine ring to pyridine-N-oxide. In another example, an oxidative metabolite is formed by demethylation of a methoxy group to result in a hydroxyl group.

### Pharmaceutical Compositions

The compositions and methods of the present application may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the application and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the application. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the application. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In some embodiments, the pharmaceutically acceptable carrier comprises more than 90%, more than 80%, more than 70%, more than 60%, more than 50%, more than 40%, more than 30%, more than 20%, more than 10%, more than 9%, more than 8%, more than 6%, more than 5%, more than 4%, more than 3%, more than 2%, more than 1%, more than 0.5%, more than 0.4%, more than 0.3%, more than 0.2%, more than 0.1%, more than 0.09%, more than 0.08%, more than 0.07%, more than 0.06%, more than 0.05%, more than 0.04%, more than 0.03%, more than 0.02%, more than 0.01%, more than 0.009%, more than 0.008%, more than 0.007%, more than 0.006%, more than 0.005%, more than 0.004%, more than 0.003%, more than 0.002%, more than 0.001%, more than 0.0009%, more than 0.0008%, more than 0.0007%, more than 0.0006%, more than 0.0005%, more than 0.0004%, more than 0.0003%, more than 0.0002%, or more than 0.0001% of the pharmaceutical composition by w/w, w/v or v/v.

In some embodiments, the concentration of the compound of formula (I) or (Ia) (e.g., fexuprazan), or a pharmaceutically acceptable salt of the compound according to formula (I) or (Ia) (e.g., fexuprazan) in the composition comprises less than 100%, less than 90%, less than 80%, less than 70%, less than 60%, less than 50%, less than 40%, less than 30%, less than 20%, less than 10%, less than 9%, less than 8%, less than 6%, less than 5%, less than 4%, less than 3%, less than 2%, less than 1%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001% of the pharmaceutical composition by w/w, w/v or v/v.

In some embodiments, the concentration of the compound of formula (I) or (Ia) (e.g., fexuprazan), or a pharmaceutically acceptable salt of the compound according to formula (I) or (Ia) (e.g., fexuprazan) is in the range of about 0.0001% to about 50%, about 0.001% to about 40%, about 0.01% to about 20%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% of the pharmaceutical composition by w/w, w/v or v/v.

In some embodiments, the concentration of the compound of formula (I) or (Ia) (e.g., fexuprazan), or a pharmaceutically acceptable salt of the compound according to formula (I) or (Ia) (e.g., fexuprazan) is in the range of about 0.0001% to about 5%, about 0.001% to about 4%, about 0.01% to about 2%, about 0.02% to about 1%, or about 0.05% to about 0.5% of the pharmaceutical composition by w/w, w/v or v/v.

Methods of preparing these formulations or compositions include the step of bringing into association an active compound, such as a compound of the application, with the carrier and, optionally, one or more accessory ingredients. In general, the formulations are prepared by uniformly and intimately bringing into association a compound of the present application with liquid carriers, or finely divided solid carriers, or both, and then, if necessary, shaping the product.

Formulations of the application suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present application as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Non-limiting examples of discrete oral dosage forms include tablets, capsules, caplets, gelatin capsules, sustained release formulations, lozenges, thin films, lollipops, chewing gum. In some embodiments, the discrete oral dosage form is an orally disintegrating oral dosage form, such as, e.g., an orally disintegrating tablet.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations suitable for sublingual administration, typically are formulated to dissolve rapidly upon placement in the mouth, allowing the active ingredient to be absorbed via blood vessels under the tongue. Exemplary sublingual formulations include, e.g., lozenges comprising the active ingredient in a flavored basis, usually sucrose and acacia or tragacanth; pastilles comprising the active ingredient in an inert basis such as gelatin and glycerin, or sucrose and acacia; mouthwashes comprising the active ingredient in a suitable liquid carrier; orally disintegrating tablets which may, for example, disintegrate in less than 90 seconds upon placement in the mouth; and thin films. Such disintegration can be measured by an in vitro dissolution test. Formulations for buccal administration can include, e.g., buccal tablets, bioadhesive particles, wafers, lozenges, medicated chewing gums, adhesive gels, patches, films, which may be delivered as an aqueous solution, a paste, an ointment, or aerosol, to name a few. Formulations suitable for intrapulmonary or nasal administration can have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of cancerous infections as described below. A pharmacological formulation of the present application can be administered to the patient in an injectable formulation containing any compatible carrier, such as various vehicle, adjuvants, additives, and diluents; or compositions utilized in the present application can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present application include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present application to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this application. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the application include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this application, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

In some embodiments, administration may comprise infusion. In some cases, infusion may involve chronic, steady dosing. Devices for chronic, steady dosing, e.g., by a controlled pump, are known in the art, (examples may be described in U.S. Pat. Nos. 7,341,577, 7,351,239, 8,058,251, herein incorporated by reference).

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the application. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the application will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present application, the active compound may be administered two, three, or four times daily. In preferred embodiments, the active compound will be administered once daily.

In certain embodiments of any of the foregoing methods, the pharmaceutical composition is administered to the subject chronically. In other embodiments of any of the foregoing methods, the pharmaceutical composition is administered to the subject acutely.

In certain embodiments of any of the foregoing methods, the pharmaceutical composition is administered to the subject for at least 6 days. In certain such embodiments, the pharmaceutical composition is administered to the subject for at least 7 days. In certain embodiments, the subject for at least four weeks. In certain embodiments, the pharmaceutical composition is administered to the subject for at least 12 weeks.

The patient receiving this treatment is any animal in need, including primates, in particular humans, and other mammals such as equines, cattle, swine and sheep; and poultry and pets in general.

This application includes the use of pharmaceutically acceptable salts of compounds of the application in the compositions and methods of the present application. The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, such as an amine, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, camphorsulfonic and the like. In certain embodiments, the pharmaceutically acceptable salt is a hydrochloride salt. In certain embodiments, the pharmaceutically acceptable salt is a camsylate salt. In certain embodiments, contemplated salts of the compounds include, but are not limited to, alkyl, dialkyl, trialkyl or tetra-alkyl ammonium salts. In certain embodiments, contemplated salts of compounds include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of compounds include, but are not limited to, Li, Na, Ca, K, Mg, Zn or other metal salts. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The compounds of the application, including their pharmaceutically acceptable salts, can also exist as various solvates, such as with water (also known as hydrates), methanol, ethanol, dimethylformamide, diethyl ether, acetamide, and the like. Mixtures of such solvates can also be prepared. The source of such solvate can be from the solvent of crystallization, inherent in the solvent of preparation or crystallization, or adventitious to such solvent.

The compounds of the application, including their pharmaceutically acceptable salts, can also exist as various polymorphs, pseudo-polymorphs, or in amorphous state. As used herein, the term “polymorph” refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus crystalline polymorphic forms are important aspects in the development of suitable dosage forms in pharmaceutical industry.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the application comprises a method for conducting a pharmaceutical business, by determining an appropriate formulation and dosage of a compound of the application for treating or preventing any of the diseases or conditions as described herein, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the application relates to a method for conducting a pharmaceutical business by determining an appropriate formulation and dosage of a compound of the application for treating or preventing any of the disease or conditions as described herein, and licensing, to a third party, the rights for further development and sale of the formulation.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject application have been discussed, the above specification is illustrative and not restrictive. Many variations of the subject of the application will become apparent to those skilled in the art upon review of this specification and the claims below. The full scope of the application should be determined by reference to the claims, along with their full scope of equivalents, and the specification, along with such variations.

1. A method of treating short bowel syndrome or a pathological hypersecretory condition in a subject, comprising administering to a subject in need thereof an effective amount of a compound of formula (I), 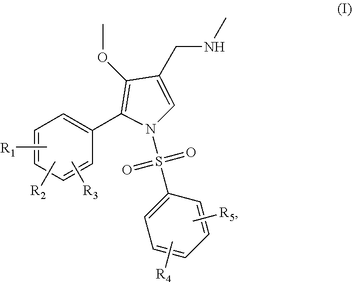
or a pharmaceutically acceptable salt thereof, wherein:
R₁, R₂ and R₃ are each independently selected from hydrogen and halogen,
R₄ and R₅ are each independently selected from hydrogen, halogen, C₁₋₄alkyl, C₁₋₄alkoxy, C₁₋₄haloalkyl, and C₁₋₄haloalkoxy.

2. The method of claim 1, wherein the compound of formula (I), or a pharmaceutically acceptable salt thereof, comprises a compound of formula (Ia), 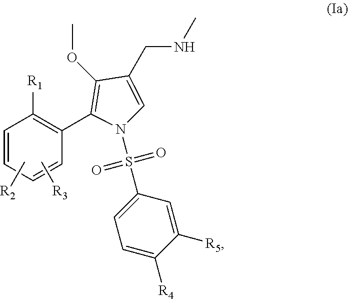
or a pharmaceutically acceptable salt thereof, wherein R₁-R₄ are as defined in claim 1.

3. The method of claim 1, wherein R₁, R₂ and R₃ are not simultaneously hydrogen.

4. The method of claim 1, wherein R₁, R₂ and R₃ are each independently selected from hydrogen, fluoro, and chloro.

5. The method of claim 1, wherein R₁ is halogen, and R₂ and R₃ are each independently selected from hydrogen and halogen.

6. The method of claim 5, wherein R₁ is fluoro, and R₂ and R₃ are each independently selected from hydrogen, fluoro, and chloro.

7. The method of claim 5, wherein R₁ is chloro, and R₂ and R₃ are each independently hydrogen.

8. The method of claim 1, wherein R₄ and R₅ are not simultaneously hydrogen.

9. The method of claim 1, wherein R₄ and R₅ are each independently selected from hydrogen, chloro, fluoro, methyl, trifluoromethyl, methoxy, and difluoromethoxy.

10. The method of claim 9, wherein R₄ is hydrogen and R₅ is selected from chloro, fluoro, methyl, trifluoromethyl, methoxy, and difluoromethoxy.

11. The method of claim 9, wherein R₄ and R₅ are each independently selected from chloro and fluoro.

12. The method of claim 1, wherein R₁ is fluoro, R₂ and R₃ are each independently selected from hydrogen and fluoro, R₄ is hydrogen, and R₅ is selected from chloro and trifluoromethyl.

13. A method of treating short bowel syndrome or a pathological hypersecretory condition in a subject, comprising administering to a subject in need thereof an effective amount of fexuprazan, 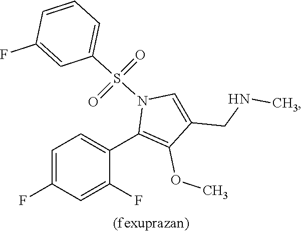 [SMILES: CNCc1cn(S(=O)(=O)c2cccc(F)c2)c(-c2ccc(F)cc2F)c1OC]
or a pharmaceutically acceptable salt thereof.

14. The method of claim 13, wherein the fexuprazan, or a pharmaceutically acceptable salt thereof, is fexuprazan hydrochloride.

15. The method of claim 1, wherein the method comprises a method of treating a pathological hypersecretory condition.

16. The method of claim 15, wherein the pathological hypersecretory condition comprises gastric acid hypersecretion.

17. The method of claim 16, wherein the pathological hypersecretory condition comprises Zollinger-Ellison syndrome.

18. The method of claim 16, wherein the pathological hypersecretory condition comprises idiopathic gastric acid hypersecretion.

19. The method of claim 16, wherein the pathological hypersecretory condition comprises hypergastrinemia.

20. Use of a compound of compound of formula (I), 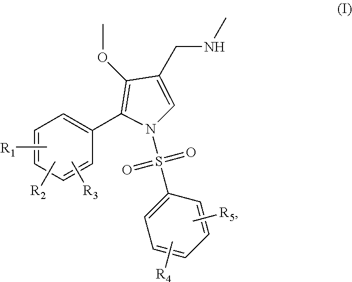
or a pharmaceutically acceptable salt thereof, wherein:
R₁, R₂ and R₃ are each independently selected from hydrogen and halogen,
R₄ and R₅ are each independently selected from hydrogen, halogen, C₁₋₄alkyl, C₁₋₄alkoxy, C₁₋₄haloalkyl, and C₁₋₄haloalkoxy
in the preparation of a medicament for the treatment of short bowel syndrome or a pathological hypersecretory condition.

21. The use claim 20, wherein the medicament is for the treatment of a pathological hypersecretory condition.

22. The use of claim 21, wherein the pathological hypersecretory condition comprises gastric acid hypersecretion.

23. The use of claim 22, wherein the pathological hypersecretory condition is Zollinger-Ellison syndrome.

24. The method of claim 22, wherein the pathological hypersecretory condition comprises idiopathic gastric acid hypersecretion.

25. The method of claim 22, wherein the pathological hypersecretory condition comprises hypergastrinemia.
